(19) United States
(12) Patent Application Publication
(10) Pub. No.: US 2005/0080374 A1
(43) Pub. Date: Apr. 14, 2005

(54) Method and apparatus for treating acute myocardial infarction with selective hypothermic perfusion

(76) Inventors: Brady Esch, San Jose, CA (US); Hoa Nguyen, San Jose, CA (US); Huu Nguyen, San Jose, CA (US); Janine Robinson, Half Moon Bay, CA (US); Manny Javier, Santa Clara, CA (US)

Correspondence Address: FULWIDER PATTON LEE & UTECHT, LLP, 200 OCEANGATE, SUITE 1550, LONG BEACH, CA, 90802, US

(21) Appl. No.: 10/897,498

(22) Filed: Jul. 23, 2004

Related U.S. Application Data
(63) Continuation-in-part of application No. 10/102,124, filed on Mar. 19, 2002, now abandoned.
Application No. 10/102,124 is a continuation-in-part of application No. 09/384,467, filed on Aug. 27, 1999, now Pat. No. 6,673,040.
(60) Provisional application No. 60/098,727, filed on Sep. 1, 1998.

(51) Int. Cl. A61M 37/00

(52) U.S. Cl. 604/6.13

(57) ABSTRACT

The present invention provides an apparatus and method for induction of therapeutic hypothermia of the heart by selective hypothermic perfusion of the myocardium through the patient's coronary arteries. The apparatus consists of a guiding catheter into which blood is drawn from the aorta, directed over a heat exchanger and expelled directly into a coronary artery.

### CROSS REFERENCE TO OTHER APPLICATIONS

This application is a continuation-in-part of U.S. patent application Ser. No. 10/102,124, filed Mar. 19, 2002, which is a continuation-in-part of U.S. patent application Ser. No. 09/384,467, filed on Aug. 27, 1999, which claims the benefit of U.S. provisional application Ser. No. 60/098,727, filed on Sep. 1, 1998, the specifications of which are hereby incorporated in their entirety.

### FIELD OF INVENTION

The present invention relates generally to methods and devices for treatment of heart disease. More particularly, it relates to methods and devices for treating acute myocardial infarction with selective hypothermic perfusion.

### BACKGROUND OF THE INVENTION

Heart disease is the most common cause of death in the United States and in most countries of the western world. Coronary artery disease accounts for a large proportion of the deaths due to heart disease. Coronary artery disease is a form of atherosclerosis in which lipids, cholesterol and other materials deposit in the arterial walls gradually narrowing the arterial lumen, thereby depriving the myocardial tissue downstream from the narrowing of blood flow that supplies oxygen and other critical nutrients and electrolytes. These conditions can be further exacerbated by a blockage due to thrombosis, embolization or arterial dissection at the site of the stenosis. A severe reduction or blockage of blood flow can lead to ischemia, myocardial infarction and necrosis of the myocardial tissue.

Recent research has indicated that, during the acute stages of myocardial infarction, as much as half of the myocardial tissue at risk can be salvaged by hypothermic treatment of the ischemic area. It is theorized that hypothermia retards the impact of reperfusion injury and may halt the progression of apoptosis, or programmed cell death. To date, most attempts at hypothermic treatment for acute myocardial infarction have involved global hypothermia of the patient's entire body, for example using a blood heat exchanger inserted into the patient's vena cava. While this method has shown some efficacy in initial trials, it has a number of drawbacks. In particular, the need to cool the patient's entire body with the heat exchanger slows the process and delays the therapeutic effects of hypothermia. The more quickly the patient's heart can be cooled, the more myocardial tissue can be successfully salvaged. Global hypothermia has another disadvantage in that it can trigger shivering in the patient. A number of strategies have been devised to stop the patient from shivering, but these add to the complexity of the procedure and have additional risk associated with them. Shivering can be avoided altogether by induction of localized hypothermia of the heart or of the affected myocardium without global hypothermia. Localized hypothermia has the additional advantage that it can be achieved quickly because of the lower thermal mass of the heart compared to the patient's entire body. Rapid induction of therapeutic hypothermia gives the best chance of achieving the most myocardial salvage and therefore a better chance of a satisfactory recovery of the patient after acute myocardial infarction.

In addition to the desirability of rapidly cooling the affected myocardium, it is most desirable to be able to simultaneously perform any of various interventional procedures that may be appropriate without interrupting or compromising the ability to continue to cool the myocardium. Reliance on vascular access to perform such functions simultaneously has to date been precluded due to the space limitations inherent in the vasculature.

What would be desirable is an apparatus and method for more rapidly inducing therapeutic hypothermia of the heart or of the affected myocardium in a patient experiencing acute myocardial infarction. Additionally, it would be most desirable to be able to continuously cool the myocardium and/or maintain a reduced temperature during the positioning and deployment of interventional devices in a coronary artery as well as during the performance of interventional procedures.

### SUMMARY OF THE INVENTION

In keeping with the foregoing discussion, the present invention provides an apparatus and method for inducing therapeutic hypothermia of the heart by selective hypothermic perfusion of the myocardium through the patient's coronary arteries. The apparatus and method provide rapid cooling of the affected myocardium to achieve optimal myocardial salvage in a patient experiencing acute myocardial infarction. Additionally, the device allows for uninterrupted cooling while interventional devices are moved into position and deployed and while interventional procedures are performed.

The apparatus takes the form of a guiding catheter that in addition to serving the functions of a conventional guiding catheter, also serves to continuously cool blood that is routed therethrough into a selected coronary artery. As such, cooling can commence as soon as the guiding catheter is in place and the need to interrupt or compromise cooling capability for interventional capability is obviated as the guiding catheter remains in place and continues to cool while serving as the primary conduit for all subsequently selected interventional devices. The time, effort and risk associated with the placement of multiple devices, in a tandem or in a sequential fashion is thereby effectively obviated.

The heat exchanger that is disposed in the guiding catheter of the present invention may rely on any of a number of different mechanisms to cool blood that flows thereover. Examples of cooling mechanisms suitable for such application include but are not limited to systems that rely on evaporative cooling, the circulation of an externally cooled medium through the heat exchanger, the expansion of a liquid and/or gas within the heat exchanger and the use of a Peltier effect device. The heat exchanger must be sufficiently small to be accommodated within a guiding catheter sized for introduction into a coronary artery while additionally allowing for the flow of blood thereover and the advancement of a guidewire or interventional devices thereby. Additionally, the temperature of the heat exchanging surface and the size of such surface must be selected so as to yield an acceptable temperature drop in the blood flowing thereover.

Any number of different mechanisms may be relied upon to draw blood from the aorta into the catheter, to direct the flow of blood over the heat exchanger and to expel the cooled blood into a coronary artery. Reliance on a passive mechanism such as by “autoperfusion” is preferred wherein a pressure differential that is established between the blood in the aorta and blood in the coronary artery is exploited. Such system relies on an occlusion or near occlusion that is created between the exterior of the catheter and the coronary ostium or the wall of a coronary artery. Intake ports proximal to such occlusion set the exterior of the portion of catheter located in the aorta into fluid communication with an internal lumen while an exit port distal to such occlusion sets the internal lumen into fluid communication with the interior of the coronary artery. The heat exchanger is positioned between the two ports. Any of various devices can be relied upon to create an appropriate occlusion or seal so as to prevent or restrict the flow of blood from the aorta into the coronary artery along the exterior of the catheter. The pressure differential that results automatically causes blood to be drawn in through the intake ports, to flow over the heat exchanger and into the coronary artery.

The guiding catheter of the present invention is configured for transluminal introduction via an arterial insertion site, such as a femoral, subclavian or brachial artery and may be advanced into position over a previously placed guidewire. The distal end of the catheter is configured for engaging the coronary ostium or entering into the selected coronary artery, at which point the occlusion device forms a fully occlusive or nearly fully occlusive seal between the exterior of the guiding catheter and the coronary ostium or wall of such coronary artery so as to induce autoperfusion. Alternatively, the device can be adapted to cool other organs such as for example the brain or the kidneys. The temperature of the heat exchanger may be controlled to achieve a target temperature within the myocardium whereby any number of feedback or feedforward systems may be relied upon to attain and then maintain such temperature.

These and other features of the present invention will become apparent from the following detailed description of preferred embodiments which, taken in conjunction with the accompanying drawings, illustrate by way of example the principles of the invention.

### BRIEF DESCRIPTION OF THE DRAWINGS

FIG. 1 is semi-schematic illustration of the system of the present invention placed within a patient;

FIG. 2 is an enlarged view of the distal section of the guiding catheter placed within a patient;

FIG. 3 is a greatly enlarged cross-sectional view of a preferred embodiment of the guiding catheter of the present invention;

FIG. 4 is a greatly enlarged cross-sectional view of a alternative preferred embodiment of the guiding catheter of the present invention;

FIG. 5 is a greatly enlarged cross-sectional view of a alternative preferred embodiment of the guiding catheter of the present invention;

FIGS. 6 and 6A are greatly enlarged cross-sectional views of an alternative preferred embodiment of the guiding catheter of the present invention;

FIG. 7 is a greatly enlarged cross-sectional view of the heat exchanger section of the guiding catheter of a preferred embodiment of the guiding catheter of the present invention;

FIG. 8 is a flow diagram of the heat exchanger shown in FIG. 7;

FIG. 9 is a greatly enlarged view of a heat exchanger of an alternative embodiment guiding catheter of the present invention; and

FIG. 10 is a greatly enlarged view of a heat exchanger of an alternative embodiment guiding catheter of the present invention.

### DETAILED DESCRIPTION OF PREFERRED EMBODIMENTS

The Figures illustrate preferred embodiments of the present invention directed to a therapeutic hypothermia system for quickly and efficiently reducing the temperature of a patient's myocardium. As such, the embodiments are illustrative of a system that includes a guiding catheter that is percutaneously introduced and intraluminally advanced into a coronary ostium or artery. The guiding catheter induces blood from the aorta to be drawn into an internal lumen, to flow over a heat exchanger positioned within the catheter and to be expelled into the coronary artery while simultaneously allowing for the introduction of any of various interventional devices into such artery.

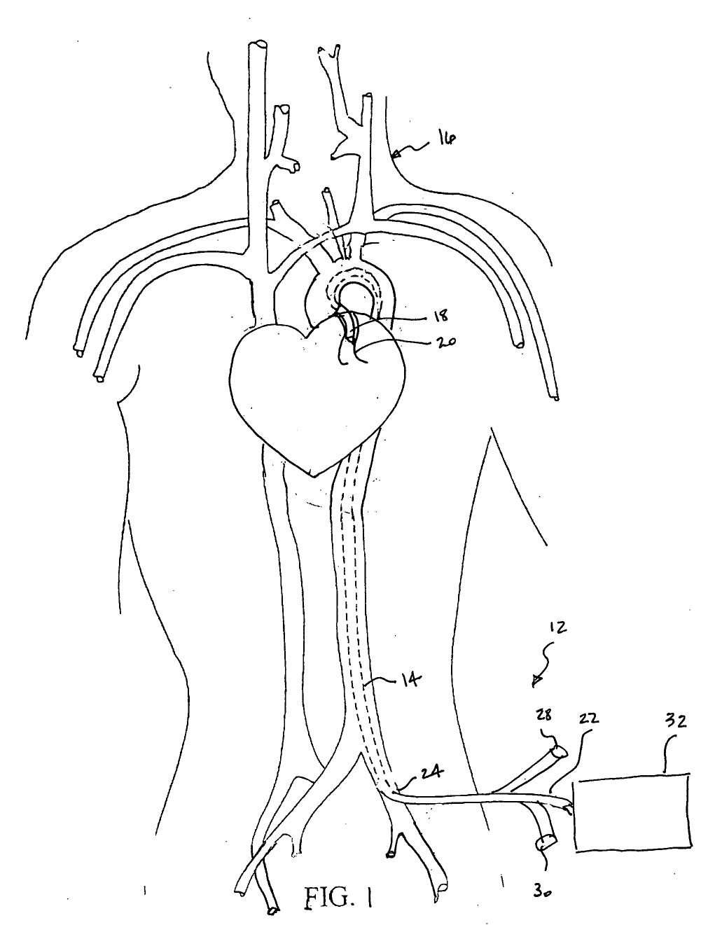

FIG. 1 is a semi-schematic illustration of the deployed therapeutic hypothermia system 12 of the present invention. A guiding catheter 14 is shown in place within a patient 16. The catheter's distal tip 18 is in position within a coronary artery 20 while its proximal end 22 is positioned outside of the patient. The insertion site of this particular embodiment is the femoral artery 24. The guiding catheter can accommodate a standard hemostasis Y-adaptor and includes ports 28, 30 through which any of various interventional devices can be introduced for advancement to and beyond the catheter's distal end. Additionally, a cooling control console 32 is shown positioned at the proximal end of the catheter. Such console serves to control the removal of heat from a heat exchanger that is positioned within the catheter near its distal end and, depending upon which form of cooling is employed, can include gas or liquid handling equipment or alternatively, means for powering a Peltier device. Additionally, the station may receive input from various sensors that monitor the effect of the cooling so that the desired effect in the myocardium can be achieved.

FIG. 2 is an enlarged cross-sectional view of the aorta 34 showing the guiding catheter 14 of the present invention in its deployed position. The catheter extends upwardly along the descending aorta 36, through the aortic arch 38 and into a coronary artery 20 near the aortic root 40. Its distal tip 18 is shown in position within the coronary artery. Visible in this view are one or more intake ports 42 through which blood flow enters the catheter and exit ports 44 at the distal end of the catheter through which blood flow is expelled into the coronary artery. Neither the heat exchanger nor an occlusion mechanism is shown in this depiction.

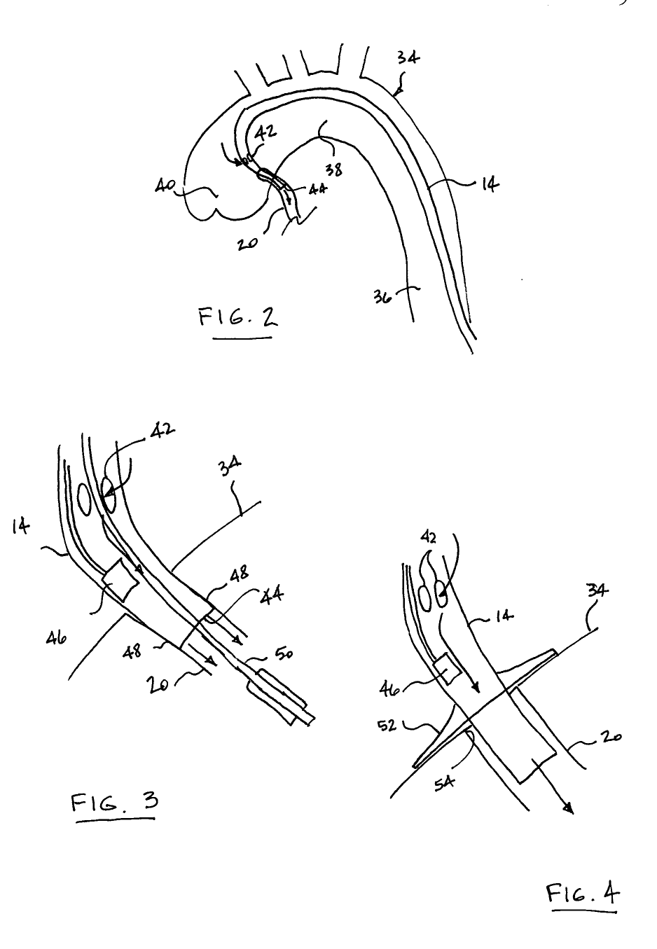

FIG. 3 is a greatly enlarged cross-sectional view of the distal section of a preferred embodiment of the guiding catheter 14 of the present invention. This illustration shows the relative placement of the intake ports 42, heat exchanger 46 and distal port 44. The heat exchanger is depicted schematically and may be positioned at any point between the intake and exit ports. In this particular embodiment, contact between the exterior of the catheter and the wall of the coronary artery at 48 is relied upon to form an occlusion or near occlusion. The seal formed thereby prevents the flow of blood between the exterior of the guiding catheter and the wall of the coronary artery and thereby creates a pressure differential between blood in the aorta and in the coronary artery. Additionally shown in this illustration is an interventional device 50 in the form of a balloon catheter that extends through the guiding catheter and into the coronary artery.

FIG. 4 is a greatly enlarged cross-sectional view of the distal section of another preferred embodiment of the guiding catheter of the present invention. This embodiment is similar to the embodiment depicted in the FIG. 3 with the exception of the occlusion mechanism that is relied upon to form a pressure differential between blood in the aorta and blood in the coronary artery. Rather than relying on the interference between the exterior of the catheter and the coronary wall, a flexible skirt 52 is fitted about the exterior of the catheter. As the catheter is advanced into the coronary artery, the skirt engages the aorta about the coronary ostium 54 and forms a seal therewith. The resulting occlusion or near occlusion causes a pressure differential to be established which causes blood to be drawn in through intake ports 42, flow over heat exchanger 46 and out through exit port 44 into the coronary artery 20.

FIG. 5 is a greatly enlarged cross-sectional view of yet another preferred embodiment of the present invention wherein the occlusion mechanism takes the form of an inflatable balloon 56 disposed about the exterior of the catheter. Upon advancement of the distal end of the guiding catheter into the coronary artery, the occlusion balloon is inflated through a lumen 58 extending to the proximal end of the catheter to a sufficiently large size so as to sealing or near sealingly engage the coronary artery wall. Blood flow between the exterior of the catheter and the artery wall is thereby precluded and the pressure differential necessary to induce autoperfusion is thereby established.

FIG. 6 is a greatly enlarged cross-sectional view of another preferred embodiment of the present invention wherein the occlusion mechanism simultaneously serves as a heat exchanger. The occlusion mechanism/heat exchanger takes the form of an inflatable balloon 60 fitted about the exterior of the catheter. A supply line 62 and return line 64 serve to route coolant through the balloon. By restricting the flow in the return line, the balloon becomes inflated while coolant is continuously cycled therethrough. The cooling and flow rate of the coolant is controlled by the cooling control console 32 at the proximal end of the catheter. Any of a number of suitable fluids can be employed, including a saline solution or CO₂ in either its liquid or gaseous phase or both phases wherein the CO₂ undergoes expansion from its liquid to its gaseous phase. Upon inflation of the balloon, an occlusion or near occlusion is formed between the exterior of the catheter and the artery wall to establish the requisite pressure differential.

FIG. 6A illustrates an alternative deployment of the device shown in FIG. 6. Positioning of the balloon just outside of the ostium can similarly be relied upon to occlude or restrict the flow of blood between the catheter and the arterial wall. The resulting pressure differential serves to induce the desired autoperfusion effect.

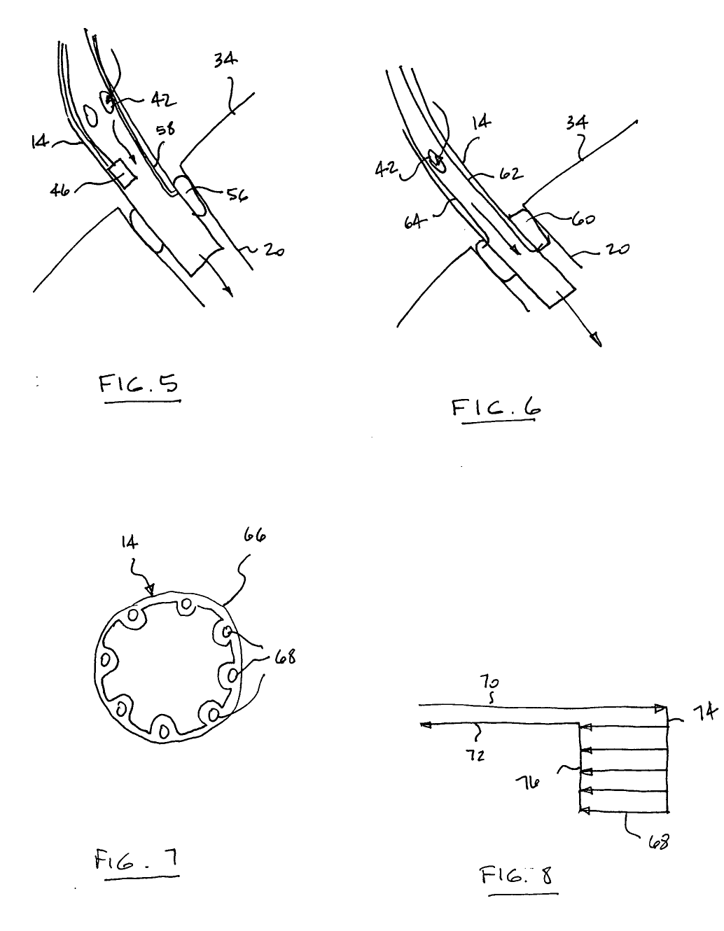

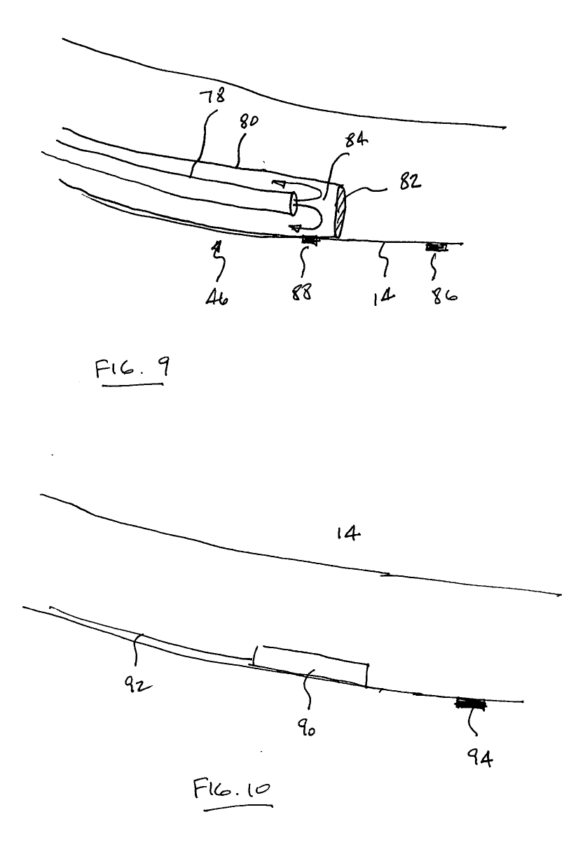

FIG. 7 is a greatly enlarged cross-sectional view of a preferred embodiment of the present invention wherein the guiding catheter 14 includes a section of cooling lumens 68 that are incorporated in the catheter wall that serve as a heat exchanger 46. A flow diagram is shown in FIG. 8 wherein a supply line 70 and return line 72 extend along the length of the catheter, preferably incorporated in the catheter wall. The supply line conducts coolant to a distribution manifold 74 that supplies the individual cooling lumens 68 while a collection manifold 76 routes the coolant to the return line. The cooling and flow rate of the coolant is controlled by the cooling control console 32 at the proximal end of the catheter. Any of a number of suitable fluids can be employed, including a saline solution or CO₂ in either its liquid or gaseous phase or both phases wherein the CO₂ undergoes expansion from its liquid to its gaseous phase.

FIG. 9 is a greatly enlarged cross-sectional view of a preferred embodiment of a heat exchanger 46 that is accommodated within, on the side or in the wall of the guiding catheter 14. A supply lumen 78 is accommodated within a return lumen 80 wherein the distal end 82 of the return lumen is sealed and the offset between the distal ends of the two lumens serves as an expansion chamber. Fluid in its gaseous or liquid form is expelled from the distal end of the supply lumen at which point it expands and loses temperature. The exterior of the distal section of the return lumen may be finned or otherwise configured for high surface area to promote the transfer of heat from blood flowing thereover to the cooled gas flowing in a proximal direction in the annular space between the two lumens. The pressure and flow rate of the fluid is controlled by the appropriate valving in the cooling control console 32 situated at the proximal end of the catheter. Temperature sensors 86 and 88 may be incorporated in the catheter to provide feedback as to the efficacy of the cooling operation. Sensor 86 may be relied upon to measure the temperature of the cooled blood while sensor 88 would provide temperature data for the heat exchanger. Temperature sensors to measure the temperature of the cooled blood may also be incorporated into other interventional devices and used in conjunction with the guiding catheter. Suitable gasses for such application include but are not limited to CO₂ and N₂₀O.

FIG. 10 is an enlarged cross-sectional view of an alternative preferred embodiment of the present invention in which the heat exchanger comprises a Peltier device. Electrical conduits extend from the cooling supply station situated outside of the patient at the proximal end of the catheter to the Peltier device. The Peltier device has a cooling side 96 positioned to contact the blood flowing within the guiding catheter and a warming side 98 that contacts the blood flowing with the aortic root, preferably in a location that is unlikely to supply the intake ports 42. The device may include fins to promote the transfer of heat thereto from the blood flowing thereover. A temperature sensor 94 downstream from the heat exchanger may be relied upon to monitor the efficacy of the device and allow the power supplied thereto to be controlled.

While particular forms of the invention have been described and illustrated, it will also be apparent to those skilled in the art that various modifications can be made without departing from the spirit and scope of the invention. More particularly, the illustrated and described embodiments can be adapted and appropriately deployed to cool other end organs such as the brain or the kidneys. Accordingly, it is not intended that the invention be limited except by the appended claims.

1. A therapeutic hypothermia system, comprising:
a guiding catheter configured for introduction into a patient's vasculature, having a distal end configured for advancement into an artery;
an occlusion mechanism configured for limiting blood flow between said guiding catheter and a wall of said artery;
a flow path for blood extending from a point on said guiding catheter's exterior proximal to said occlusion device, through said guiding catheter to a point on said guiding catheter's exterior distal to said occlusion device; and
a heat exchanger positioned in said flow path for reducing the temperature of blood flowing therethrough.

2. The therapeutic hypothermia system of claim 1, wherein said artery comprises a coronary artery.

3. The therapeutic hypothermia system of claim 1, wherein said artery comprises a renal artery.

4. The therapeutic hypothermia system of claim 1, wherein said artery comprises a cerebral artery.

5. The therapeutic hypothermia system of claim 1, wherein said guiding catheter is configured to accommodate the advancement of interventional devices therethrough.

6. The therapeutic hypothermia system of claim 1, wherein said flowpath comprises a proximal port, an internal lumen and a distal port.

7. The therapeutic hypothermia system of claim 1, wherein said proximal port is formed in said guiding catheter so as to be located in the aorta when said distal end is positioned within said artery.

8. The hypothermia system of claim 1, wherein said occlusion mechanism comprises a dimensioning of an exterior surface of said guiding catheter so as to engage the wall of said artery and form a seal when inserted thereinto.

9. The hypothermia system of claim 1, wherein said occlusion mechanism comprises an inflatable balloon disposed about the exterior of said guiding catheter, configured so as to engage the wall of said artery or associated ostium and form a seal upon inflation.

10. The hypothermia system of claim 1, wherein said occlusion mechanism comprises a flexible skirt disposed about the exterior of said guiding catheter, configured to engage a section of aortic wall about a coronary ostium and form a seal.

11. The hypothermia system of claim 1, wherein said heat exchanger relies on a circulation of coolant therethrough.

12. The hypothermia system of claim 1, wherein said heat exchanger relies on an expansion of a gas to reduce temperature.

13. The hypothermia system of claim 1, wherein said heat exchanger relies on a phase change of a liquid to a gas.

14. The hypothermia system of claim 1, wherein said heat exchanger relies on a Peltier device to reduce temperature.
